(19) United States
(12) Patent Application Publication
(10) Pub. No.: US 2023/0172962 A1
(43) Pub. Date: Jun. 8, 2023

(54) NRTIS, NRTI METABOLITES, AND NRTI ANALOGS FOR MACULAR DEGENERATION AND VIRAL INFECTIONS

(71) Applicant: University of Virginia Patent Foundation, Charlottesville, VA (US)

(72) Inventors: Jayakrishna Ambati, Charlottesville (US); Kameshwari Ambati, Charlottesville (US)

(73) Assignee: University of Virginia Patent Foundation, Charlottesville, VA (US)

(21) Appl. No.: 17/912,805

(22) PCT Filed: Mar. 19, 2021
(86) PCT No.: PCT/US2021/023180

§ 371 Date: Sep. 19, 2022

Related U.S. Application Data
(60) Provisional application No. 62/992,577, filed on Mar. 20, 2020.

(51) Int. Cl. A61K 31/7072; A61K 31/513; A61K 45/06; A61P 27/02; A61P 29/00

(52) U.S. Cl. CPC A61K 31/7072; A61K 31/513; A61K 45/06; A61P 27/02; A61P 29/00

(57) ABSTRACT

Methods for treating and/or inhibiting progression of diseases, conditions, and/or disorders. In some embodiments, the methods include administering to as subject in need thereof a composition that includes a nucleoside reverse transcriptase inhibitor (NRTIs), an NRTI metabolite, an NRTI analog, a pharmaceutically acceptable salt and/or metabolite thereof, a prodrug of an NRTI, or any combination thereof. Also provided are methods for inhibiting development of macular degeneration in subjects; methods for inhibiting development and/or progression of viral infection and/or a disease, condition, disorder, and/or symptom associated therewith in a subject in need thereof; methods for inhibiting development and/or progression of acute respiratory distress syndrome in subjects; methods for inhibiting development and/or progression of cytokine storm syndrome in subjects; methods for inhibiting development and/or progression of fibrosis in subjects, and compositions for use in the presently disclosed methods.

### CROSS REFERENCE TO RELATED APPLICATION

The presently disclosed subject matter claims the benefit of U.S. Provisional Patent Application Ser. No. 62/992,577, filed Mar. 20, 2019, the disclosure of which incorporated herein by reference in its entirety.

### GOVERNMENT INTEREST

This invention was made with government support under Grant Nos. EY028027 and EY029799 awarded by The National Institutes of Health. The government has certain rights in the invention.

### TECHNICAL FIELD

The presently disclosed subject matter relates generally to compositions and methods for treating diseases and/or disorders. In some embodiments, the compositions comprise bacterium-derived microvesicles and/or nanovesicles/nanoparticles.

### BACKGROUND

Dry (atrophic) macular degeneration affects nearly 200 million people worldwide (Wong et al., 2014). There is no FDA-approved therapy for this disease, which is the leading cause of irreversible blindness among people over 50 years of age (Mitchell et al., 2018). Vision loss in this condition results from degeneration of the retinal pigmented epithelium (RPE). RPE cell death in dry macular degeneration is driven by accumulation of toxic molecules known as Alu RNAs, which are noncoding RNAs in the human genome (Kaneko et al., 2011). Alu RNA induces RPE degeneration by activating a macromolecular protein complex known as the nucleotide-binding domain and leucine-rich repeat pyrin domain-3 (NLRP3) inflammasome (Tarallo et al., 2012). Wet (neovascular) macular degeneration can be treated with intraocular injection of drugs that inhibit vascular endothelial growth factor-A (VEGF-A); however, long-term treatment outcomes are unsatisfying. We previously demonstrated that nucleoside reverse transcriptase inhibitors (NRTIs) were effective in animal models of dry (Fowler et al., 2014) and wet (Mizutani et al., 2015) macular degeneration, by virtue of their ability to block NLRP3 inflammasome activation and inhibit the P2X7 receptor, respectively.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for treating and/or inhibiting progression of diseases, conditions, and/or disorders in subjects suffering from and/or at risk for developing a disease, condition, and/or disorder. In some embodiments, the methods comprise, consist essentially of, or consist of administering to the subject a composition comprising a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof, in an amount and via a route effective for treating and/or inhibiting progression of the disease, condition, and/or disorder in the subject. In some embodiments, the disease, condition, and/or disorder comprises wet macular degeneration, dry macular degeneration, a viral infection, acute respiratory distress syndrome, cytokine storm syndrome, fibrosis (e.g., subretinal fibrosis), and/or any combination thereof. In some embodiments, the composition further comprises a pharmaceutically acceptable carrier, diluent, or excipient, optionally a pharmaceutically acceptable carrier, diluent, or excipient that is pharmaceutically acceptable for use in a human. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT; 3′-azido-2′,3′-dideoxythymidine), derivatives thereof, optionally alkylated derivatives thereof, further optionally tri-methoxy-3TC and/or di-ethoxy-AZT and/or optionally an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

In some embodiments, the presently disclosed methods further comprise, consist essentially of, or consist of administering to the subject an additional treatment. In some embodiments, the additional treatment is a treatment designed to treat or reduce the progression of wet macular degeneration, dry macular degeneration, a viral infection, optionally wherein the viral infection is with a corona virus, further optionally SARS-CoV and/or SARS-CoV-2, acute respiratory distress syndrome, cytokine storm syndrome, fibrosis (e.g., subretinal fibrosis), and/or any combination thereof.

In some embodiments, the presently disclosed subject matter also relates to methods for inhibiting development of macular degeneration in subject at risk therefor. In some embodiments, the methods comprise, consist essentially of, or consist of administering to the subject a composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for treating and/or inhibiting development of macular degeneration in the subject. In some embodiments, the composition further comprises a pharmaceutically acceptable carrier, diluent, and/or excipient, optionally wherein the composition is pharmaceutically acceptable for use in a human. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

In some embodiments, the presently disclosed methods further comprise, consist essentially of, or consist of administering to the subject an additional treatment. In some embodiments, the additional treatment is an additional treatment for wet macular degeneration, dry macular degeneration, and/or fibrosis (e.g., subretinal fibrosis).

In some embodiments, the presently disclosed subject matter also relates to methods for inhibiting a viral infection, optionally a viral infection with a coronavirus, further optionally a viral infection with SARS-CoV or SARS-CoV-2. In some embodiments, the methods comprise, consist essentially of, or consist of administering to a subject in need thereof a composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for inhibiting a viral infection in the subject. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

In some embodiments, the presently disclosed methods further comprise, consist essentially of, or consist of administering to the subject an additional treatment. In some embodiments, the additional treatment is selected from the group consisting of treatment with one or more additional antivirals.

The presently disclosed subject matter also relates in some embodiments to compositions for use in treating and/or inhibiting development and/or progression of macular degeneration in subjects suffering from and/or at risk for developing macular degeneration. In some embodiments, the composition comprise, consist essentially of, or consist of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount effective for treating and/or inhibiting development and/or progression of the macular degeneration in the subject.

The presently disclosed subject matter also relates in some embodiments to compositions for use in treating and/or inhibiting development and/or progression of a viral infection and/or a disease, condition, disorder, and/or symptom associated therewith in a subject suffering from and/or at risk for developing viral infection and/or the disease, condition, disorder, or symptom associated therewith, the composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount effective for treating and/or inhibiting development and/or progression of the viral infection and/or the disease, condition, disorder, and/or symptom associated therewith in the subject. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

In some embodiments, the compositions for use of the presently disclosed subject matter further comprise, consist essentially of, or consist of one or more additional active agents. In some embodiments, the one or more additional active agents are agents appropriate for treating macular degeneration and/or an antiviral.

The presently disclosed subject matter also relates in some embodiments to pharmaceutical compositions for treating and/or inhibiting development and/or progression of macular degeneration in subjects in need thereof, and/or for inhibiting development and/or progression of viral infection and/or a disease, condition, disorder, and/or symptom associated therewith in subjects in need thereof, and/or for treating and/or inhibiting the development and/or progression of acute respiratory distress syndrome, cytokine storm syndrome, and/or fibrosis (e.g., subretinal fibrosis) in subjects in need thereof. In some embodiments, the pharmaceutical composition comprise, consist essentially of, or consist of an effective amount of one or more a nucleoside reverse transcriptase inhibitors (NRTIs), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof.

The presently disclosed subject matter also relates in some embodiments to compositions for preparation of medicaments for treating and/or inhibiting development and/or progression of macular degeneration in subjects in need thereof, and/or for inhibiting development and/or progression of viral infection and/or a disease, condition, disorder, and/or symptom associated therewith in subjects in need thereof, and/or for treating and/or inhibiting the development and/or progression of acute respiratory distress syndrome, cytokine storm syndrome, and/or fibrosis (e.g., subretinal fibrosis) in subjects in need thereof. In some embodiments, the pharmaceutical composition comprises, consists essentially of, or consists of an effective amount of one or more a nucleoside reverse transcriptase inhibitors (NRTIs), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof.

The presently disclosed subject matter also relates in some embodiments to methods for treating and/or inhibiting the development and/or progression of acute respiratory distress syndrome, cytokine storm syndrome, and/or fibrosis (e.g., subretinal fibrosis). In some embodiments, the methods comprise, consist essentially of, or consist of administering to a subject in need thereof a composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for treating and/or inhibiting the development and/or progression of acute respiratory distress syndrome, cytokine storm syndrome, and/or fibrosis (e.g., subretinal fibrosis) in the subject. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments. the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

In some embodiments, the methods of the presently disclosed subject matter further comprise, consist essentially of, or consist of administering to the subject an additional treatment. In some embodiments, the additional treatment is selected from the group consisting of treatment with one or more additional active agents, wherein the one or more active agents are appropriate for treating and/or inhibiting the development and/or progression of acute respiratory distress syndrome, cytokine storm syndrome, and/or fibrosis (e.g., subretinal fibrosis).

The presently disclosed subject matter also relates in some embodiments to compositions for use in treating and/or inhibiting development and/or progression of acute respiratory distress syndrome, cytokine storm syndrome, and/or fibrosis (e.g., subretinal fibrosis) in subjects suffering from and/or at risk for developing acute respiratory distress syndrome, cytokine storm syndrome, and/or fibrosis (e.g., subretinal fibrosis). In some embodiments, the compositions comprise, consist essentially of, or consist of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount effective for treating and/or inhibiting development and/or progression of the viral infection and/or the disease, condition, disorder, and/or symptom associated therewith in the subject. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

In some embodiments, the compositions for use of the presently disclosed subject matter further comprise, consist essentially of, or consist of an additional active agent. In some embodiments, the additional active agent is an agent appropriate for treating acute respiratory distress syndrome, cytokine storm syndrome, and/or fibrosis (e.g., subretinal fibrosis).

Thus, it is an object of the presently disclosed subject matter to provide compositions and methods for treating, preventing, and/or inhibiting the development and/or progression of wet macular degeneration, dry macular degeneration, a viral infection, acute respiratory distress syndrome, cytokine storm syndrome, fibrosis (e.g., subretinal fibrosis), and/or any combination thereof, or any disease, condition, disorder, and/or symptom associated with wet macular degeneration, dry macular degeneration, a viral infection, acute respiratory distress syndrome, cytokine storm syndrome, fibrosis (e.g., subretinal fibrosis), and/or any combination thereof.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1 is a representative western blot showing Alu RNA-induced cleavage of pro-caspase-1 (50 kDa band) into active caspase-1 (20 kDa band), a hallmark of inflammasome activation, was inhibited by azidothymidine (AZT), its metabolite azidothymidine glucuronide (GAZT), and a further metabolite 3′-amino-3′-deoxythymidine (AMT) in mouse BMDMs. N=3.

FIG. 2 is a bar graph showing that Alu RNA-induced IL-1β release, as monitored by ELISA, from mouse BMDMs was inhibited by AZT, GAZT, AMT, and LSO. N=3 per group. Mean±SEM.

FIGS. 3A and 3B are a bar graph and a fluorescence micrograph, respectively, of the results of laser-induced choroidal neovascularization (CNV) in mice, which was inhibited by AZT, GAZT, and AMT. The top panel shows a summary of CNV volumes, mean (SEM). The bottom panel shows representative images of CNV lesions. N=20-40.

FIG. 4 is a bar graph showing that laser-induced choroidal neovascularization (CNV) in mice was inhibited by K-8 and K-9. Mean (SEM). N=20-40.

FIG. 5 is a series of fluorescence micrographs of representative flat mounts of the retinal pigmented epithelium (RPE) of mice showing that Alu RNA-induced RPE degeneration was inhibited by GAZT, AMT, and LSO. N=8-14.

FIGS. 6A and 6B present the results of experiments showing that NRTI blocked Alu RNA-induced fibrosis. FIG. 6A is a pair of fluorescence micrographs (left and middle panels) and a bar graph showing that wild type mice injected with subretinal Alu RNA after laser injury displayed a significantly larger volume of subretinal fibrosis than mice injected with PBS. FIG. 6B is a pair of fluorescence micrographs (left and middle panels) and a bar graph showing that intravitreous injection of the NRTI AZT reduced subretinal fibrosis volume in wildtype mice. Images are representative of flat mounts stained with anti-Collagen type 1 antibodies to visualize fibrosis. N=6-8.

FIG. 7 is a bar graph showing that the NRTI analog K-8 blocked Alu RNA-induced fibrosis. Intravitreous injection of the NRTI analog K-8 reduced the volume of subretinal fibrosis induced by subretinal Alu RNA after laser injury in wild-type mice. Images are representative of flat mounts stained with anti-Collagen type 1 antibodies to visualize fibrosis. N=6-8.

### DETAILED DESCRIPTION

### I. Definitions

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the term “and/or” when used in the context of a list of entities, refers to the entities being present singly or in any and every possible combination and subcombination. Thus, for example, the phrase “A, B, C, and/or D” includes A, B, C, and D individually, but also includes any and all combinations and subcombinations of A, B, C, and D. It is further understood that for each instance wherein multiple possible options are listed for a given element (i.e., for all “Markush Groups” and similar listings of optional components for any element), in some embodiments the optional components can be present singly or in any combination or subcombination of the optional components. It is implicit in these forms of lists that each and every combination and subcombination is envisioned and that each such combination or subcombination has not been listed simply merely for convenience. Additionally, it is further understood that all recitations of “or” are to be interpreted as “and/or” unless the context clearly requires that listed components be considered only in the alternative (e.g., if the components would be mutually exclusive in a given context and/or could not be employed in combination with each other).

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein. In some embodiments, a subject is a human.

It is noted that all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in the GENBANK® biosequence database, are intended to encompass homologous and variant genes and gene products from humans and other animals including, but not limited to other mammals.

The methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, hypertension and diabetes. The additional compounds can also be used to treat symptoms associated with the injury, disease, or disorder, including, but not limited to, pain and inflammation.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

With regard to administering a composition, the term “administering” as used herein refers to any method for providing a composition and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, subcutaneous administration, intravitreous administration, including via intravitreous sustained drug delivery device, intracameral (into anterior chamber) administration, suprachoroidal injection, subretinal administration, subconjunctival injection, sub-tenon administration, peribulbar administration, transscleral drug delivery, intraocular injection, intravenous injection, intraparenchymal/intracranial injection, intra-articular injection, retrograde ureteral infusion, intrauterine injection, intratesticular tubule injection, intrathecal injection, intraventricular (e.g., inside cerebral ventricles) administration, administration via topical eye drops, and the like. Administration can be continuous or intermittent. In some embodiments, a preparation can be administered therapeutically; that is, administered to treat an existing disease or condition. In some embodiments, a preparation can be administered prophylactically; that is, administered for prevention of a disease, disorder, or condition.

The term “adult” as used herein, is meant to refer to any non-embryonic or non-juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge to region, thereby converting the F(ab′)₂ dimer into an Fab₁ monomer. The Fab₁ monomer is essentially a Fab with part of the hinge region. While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “aqueous solution” as used herein can include other ingredients commonly used, such as sodium bicarbonate described herein, and further includes any acid or base solution used to adjust the pH of the aqueous solution while solubilizing a peptide.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients can be added. The term does not exclude media which have been prepared or are intended for specific uses, but which upon modification can be used for other cell types, etc.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable”, as the term is used herein, means that at least 80% of the total mass of material implanted is resorbed within one year.

The phrases “cell culture medium”, “culture medium” (plural “media” in each case), and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) can then be used in any desired way, such as to treat a disease or disorder in a subject, or to support the growth or differentiation of a second population of cells.

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

As used herein, the term “metabolite” refers to any product of metabolism, including any product, whether intermediate or end product, of a biological activity that goes on in a cell, tissue, organ, or subject. By way of example and not limitation, an “NRTI metabolite” is a molecule that subsequent to administration of an NRTI to a subject is a metabolic product that results from metabolism of the NRTI in the subject. As such, the term “metabolic product” is equivalent to the term “metabolite” as used herein.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine”, as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of any group (e.g., an H) by an alkyl, acyl, or amino group.

More particularly, as used herein the phrase “alkylated derivative” refers to a derivative of an NRTI that as compared to the structure of the parent NRTI includes one or more alkyl groups. Exemplary alkylated derivatives of NRTIs include, but are not limited to tri-methoxy-3TC (also known as Kamuvudine-9 and K-9; see U.S. Patent Application Publication No. 2016/138425, the content of which is incorporated herein by reference in its entirety) and/or di-ethoxy-AZT (also known as Kamuvudine-8 and K-8; see U.S. Patent Application Publication No. 2019/0262341, the content of which is incorporated herein by reference in its entirety).

As used herein, a “Kamuvudine” is an NRTI. Kamuvudines and methods for their synthesis are described in U.S. Pat. No. 10,294,220, which is incorporated herein by reference in its entirety.

Thus, exemplary such NRTIs and derivatives thereof include but are not limited to AZT (3′-azido-2′,3′-dideoxythymidine), its methoxy derivative (O-methyl group; me-AZT; 1-[(5R)-2-methoxymethyl)-5-(5-methyl-2,4-dioxo-1,2,3,4tetrahydropyrimidin-1-yl)oxolan-3-yl]triaza-1,2-dien-2-ium; also referred to as Kamuvudine 5; see Structure 1 of U.S. Pat. No. 10,294,220), and the N-Me-Me-AZT derivative disclosed as Structure 10 of U.S. Pat. No. 10,294,220, cyclopropylaminopurinylcyclopentene (ABC) and its methoxy variation (O-methyl group), stavudine (d4T; CAS No. 3056-17-5; 1-[(2R,5S)-5-(hydroxymethyl)-2,5 -dihydrofuran-2-yl]-5-methyl-1,2,3,4-tetrahydropyrimidine-2,4-dione), lamivudine (3TC; also called 2′3′-dideoxycytidine; CAS No. 134678-17-4; 2′,3′-dideoxy-3′-thiacytidine-4-Amino-1-[(2R,5S)-2-(hydroxymethyl)-1,3-oxathiolan-5-yl]-1,2-dihydropyrimidin-2-one) and its derivatives Me-3TC (4-amino-1-[(2R, 5S)-2-(methoxymethyl)-1,3-oxathiolan-5-yl]-1,2-dihydropyrimidin-2-one), deamino-Me-3TC (1-[(2R,5S)-2-(methoxymethyl)-1,3-oxathiolan-5-yl)-1,2,3,4-tetrahydropyrimidine-2,4-dione), 3Me-3TC (4-(dimethylamino)-1-[(2R,5S)-2-(methoxymethyl)-1,3-oxathiolan-5-yl]-1,2-dihydropyrimidin-2-one; also referred to as Kamuvudine 6 and TM-3TC; see Structure 8 of U.S. Pat. No. 10,294,220), and cordycepin (3′-deoxyadenosine; CAS No. 73-03-0; 9-(3-Deoxy-β-d-ribofuranosyl)adenine), and derivatives thereof including but not limited to methoxy-d4T (me-d4T; 1-[(2R,5S)-5-(methoxymethyl)-2,5-dihydrofuran-2-yl]-5-methyl-1,2,3,4-tetrahydropyrimidine-2,4-dione, also referred to as Kamuvudine 1), deoxy-methyl-d4T (5-methyl-1-[2R,5R)-5-methyl-2,5-dihydrofuran-2-yl]-1,2,3,4-tetrahydropyrimidine-2,4-dione), 2me-d4T (see Structure 4 of U.S. Pat. No. 10,294,220), methylene d4T (methylene d4T; 5-methyl-1-[2R)-5-methylidene-2,5-dihydrofuran-2-yl)-1,2,3,4-tetrahydropyrimidine-2,4-dione), d4T-ene (see Structure 3 of U.S. Pat. No. 10,294,220), deoxy-amino-d4T (1-[(2R)-5-(aminomethyl)-2,5-dihydrofuran-2-yl)-5-methyl-1,2,3,4-tetrahydropyrimidine-2,4-dione), Azido-d4T (1-{[(2S,5R)-5-(5-methyl-2,4-dioxo-1,2,3,4-tetrahydropyrimidin-1-yl)-2,5-dihydrofuran-2-yl]methyl}triaza-1,2-dien-2-ium), 2Me-AZT (1-[(2R,4S,5S)-4-azide-5-(methoxymethyl)oxolan-2-yl)-3,5-dimethyl-1,2,3,4-tetrahydropyrimidine-2,4-dione; also referred to herein as Kamuvudine 4), O-Me N-Et d4T (3-ethyl-1-[(2R,5S)-5-(methoxymethyl)-2,5-dihydrofuran-2-yl]-5-methyl-1,2,3,4-tetrahydropyrimidine-2,4-dione; also referred to herein as Kamuvudine 3), 2Et-AZT:1-[(2R,4S,5S)-4-azide-5-(ethoxymethyl)oxolan-2-yl)-3-ethyl-5-methyl-1,2,3,4-etrahydropyrimidine-2,4-dione; also referred to herein as Kamuvudine 8), 2Et-d4T (1-(2R,5S)-5-(ethoxymethyl)-2,5-dihydrofuran-2-yl)-3 -ethyl-5-methyl-1,2,3,4-tetrahydropyrimidine-2,4-dione; also referred to herein as Kamuvudine 7), 3Et-3TC (4-diethylamino)-1-[2-(ethoxymethyl)-1,3-oxathiolan-5-yl)-1,2-dihydropyrimidin-2-one; also referred to herein as Kamuvudine 9), and derivatives thereof including but not limited to those disclosed in U.S. Pat. No. 10,294,220, which is incorporated herein by reference in its entirety.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the terms “condition”, “disease condition”, “disease”, “disease state”, and “disorder” refer to physiological states in which diseased cells or cells of interest can be targeted with the compositions of the presently disclosed subject matter. By way of example and not limitation, a “condition”, “disease condition”, “disease”, “disease state”, and “disorder” of the presently disclosed subject matter relates to a physiological state in which the diseased cell or cells of interest are cells of the eye, optionally RPE cells.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “diagnosis” refers to detecting a risk or propensity to a condition, disease, or disorder. In any method of diagnosis exist false positives and false negatives. Any one method of diagnosis does not provide 100% accuracy.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

As used herein, the term “homologous” refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to cells, refers to a single cell of interest, or a population of cells of interest, at least partially isolated from other cell types or other cellular material with which it occurs in a culture or a tissue of origin. A sample is “substantially pure” when it is in some embodiments at least 60%, in some embodiments at least 75%, in some embodiments at least 90%, and, in certain cases, in some embodiments at least 99% free of cells or other cellular material other than the cells of interest.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels

A “receptor” is a compound that specifically or selectively binds to a ligand.

Micro-RNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

The term “polynucleotide” as used herein includes but is not limited to DNA, RNA, complementary DNA (cDNA), messenger RNA (mRNA), ribosomal RNA (rRNA), small hairpin RNA (shRNA), small nuclear RNA (snRNA), short nucleolar RNA (snoRNA), microRNA (miRNA), genomic DNA, synthetic DNA, synthetic RNA, and/or tRNA.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof” is a patient, animal, mammal, or human, who will benefit from a method or compositions of the presently disclosed subject matter.

The term “substantially pure” describes a compound, molecule, or the like, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, such as but not limited to in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

As used herein, “substantially homologous amino acid sequences” includes those amino acid sequences which have in some embodiments at least about 95% homology, in some embodiments at least about 96% homology, in some embodiments at least about 97% homology, in some embodiments at least about 98% homology, and in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is in some embodiments at least about 50%, 65%, 75%, 85%, 95%, 99%, or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: in some embodiments in 7% sodium dodecyl sulfate SDS, 0.5 M NaPO4, 1 mM EDTA at 50° C. with washing in 2×standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments in 7% SDS, 0.5 M NaPO4, 1 mM EDTA at 50° C. with washing in 0.5×SSC, 0.1% SDS at 50° C.; and in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a,b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

As used herein, the term “transfection” as used herein refers to the introduction of a foreign nucleic acid into a cell using recombinant DNA technology. The term “transformation” means the introduction of a “foreign” (i.e., extrinsic or exogenous) gene, DNA, or RNA sequence to a host cell, such that the host cell will express the introduced gene or sequence to produce a desired substance, such as a protein or enzyme, coded by the introduced gene or sequence. The introduced gene or sequence can also be called a “cloned”, “foreign”, or “heterologous” gene or sequence or a “transgene”, and can include regulatory and/or control sequences, such as start, stop, promoter, signal, secretion, or other sequences used by a cell's genetic machinery. The gene or sequence can include nonfunctional sequences or sequences with no known function. A host cell that receives and expresses introduced DNA or RNA has been “transformed” and is a “transformant” or a “clone”, and is “transgenic”. The DNA or RNA introduced to a host cell can come from any source, including cells of the same genus or species as the host cell, or cells of a different genus or species

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

The terms “treatment” and “treating” as used herein thus refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

### II. Representative Embodiments

Disclosed herein is the discovery that certain metabolites of nucleoside reverse transcriptase inhibitors (NRTIs) that have been detected in patients taking NRTIs possess anti-NLRP3 inflammasome and anti-cytokine inhibitory ability in cells and inhibit retinal degeneration in an animal model of atrophic macular degeneration and angiogenesis in an animal model of neovascular macular degeneration. These metabolites therefore represent a prevention and/or treatment strategy, such as but not limited to administration to a subject in need thereof in an oral form and/or in a sustained release intraocular form, for both atrophic macular degeneration and neovascular macular degeneration. They could also be employed for a variety of inflammasome-driven diseases, disorders, and conditions including but not limited to viral infections such as Covid-19.

In some embodiments, the presently disclosed subject matter also relates to methods and uses of the compositions disclosed herein for treating and/or preventing development of diseases, disorders, and or conditions that are associated with NLRP3-ASC inflammasome activity in subjects. In some embodiments, the presently disclosed subject matter methods and uses comprise, consist essentially of, or consist of administering to a subject in need thereof an effective amount of fluoxetine, optionally wherein the fluoxetine is present in an enantiomerically pure form. In some embodiments, the presently disclosed subject matter relates to administering a composition to a site associated with NLRP3-ASC inflammasome activation. As used herein, the phrase “associated with NLRP3-ASC inflammasome activation” refers to any disease, disorder, and/or condition at least one symptom of consequence of which results either directly or indirectly from NLRP3-ASC inflammasome activation and for which inhibition of NLRP3-ASC inflammasome activation would be expected to ameliorate or eliminate the at least one symptom of consequence.

Various diseases are known to be associated with NLRP3-ASC inflammasome activation, and include but are not limited to arthritis, optionally rheumatoid arthritis and/or reactive arthritis, diabetes mellitus, chronic obstructive pulmonary disease, inflammatory bowel disease, irritable bowel syndrome, Duchenne muscular dystrophy, graft-versus-host disease, chronic pain, proliferative vitreoretinopathy, glaucoma, multiple sclerosis, bipolar disorder, major depressive disorder, renal fibrosis, nephritis, pulmonary fibrosis, subretinal fibrosis, Huntington's disease, osteoporosis, chronic lymphocytic leukemia, anxiety disorders, pulmonary tuberculosis, osteoporosis, optionally osteoporosis in post-menopausal women and/or fracture patients, systemic lupus erythematosus, discoid lupus erythematosus, chronic inflammatory and neuropathic pain, autosomal dominant polycystic kidney disease, spinal cord injury, Alzheimer's disease, neuropathic pain, hypertension, varicose veins, type I diabetes, type II diabetes, gout, autoimmune hepatitis, graft vascular injury, atherosclerosis, thrombosis, metabolic syndrome, salivary gland inflammation, traumatic brain injury, ischemic heart disease, ischemic stroke, Parkinson's disease, melanoma, neuroblastoma, prostate, breast, skin, and thyroid cancers, tubular early gastric cancer, neuroendocrine cancer, mucoid colon cancer, colon cancer; high-grade urothelial carcinoma, kidney clear cell carcinoma, undifferentiated ovary carcinoma, papillary intracystic breast carcinoma, gram negative sepsis, infectious Pseudomonas aeruginosa, Vibrio cholera, Legionella spp., Francisella spp., and Leishmania spp. Chlamydia spp., cryopyrinopathies; keratitis, acne vulgaris, Crohn's disease, ulcerative colitis, insulin resistance, obesity, hemolytic-uremic syndrome, polyoma virus infection, immune complex renal disease, acute tubular injury, lupus nephritis, familial cold autoinflammatory syndrome, Muckle-Wells syndrome and neonatal onset multisystem inflammatory disease, chronic infantile neurologic cutaneous and articular autoinflammatory diseases, renal ischemia-perfusion injury, glomerulonephritis, cryoglobulinemia, systemic vasculitides, IgA nephropathy, malaria, helminth parasites, septic shock, allergic asthma, hay fever, drug-induced lung inflammation, contact dermatitis, leprosy, Burkholderia cenocepacia infection, respiratory syncitial virus infection, psoriasis, scleroderma, cystic fibrosis, syphilis, Sjogren's syndrome, inflammatory joint disease, non-alcoholic fatty liver disease, sterile liver inflammation, cardiac surgery (peri-/post-operative inflammation), acute and chronic organ transplant rejection, acute and chronic bone marrow transplant rejection, tumor angiogenesis, amyotrophic lateral sclerosis, and autism spectrum disorder.

Inflammasomes require the adapter protein apoptosis associated speck-like protein containing a CARD (ASC) for the activation of caspase-1. After inflammasome activation, ASC assembles into a large protein complex, which is termed a “speck”. ASC specks can be observed as they reach a size of around 1 μm and in most cells only one speck forms upon inflammasome activation.

Thus, in some embodiments NLRP3-ASC inflammasome activation can result in ASC speck formation, and as such, the compositions of the presently disclosed subject matter can in some embodiments be employed to inhibit ASC speck formation in cells. Therefore, in some embodiments the compositions of the presently disclosed subject matter that comprise, consist essentially of, or consist of fluoxetine are employed to treat and/or prevent development of a disease, disorder, or condition associated with NLRP3-ASC inflammasome activation in a subject.

Accordingly, the presently disclosed subject matter also provides uses of compositions comprising, consisting essentially of, or consisting of fluoxetine, optionally substantially enantiomerically pure formulations thereof for treating and/or preventing development of diseases, disorder, and/or conditions in subjects in need thereof, optionally diseases, disorder, and/or conditions of the eye, further optionally diseases, disorder, and/or conditions of the retina, further optionally diseases, disorder, and/or conditions of the RPE, wherein the disease or disorder of the eye is associated with RPE degeneration. In some embodiments, a composition comprising, consisting essentially of, or consisting of fluoxetine is used to treat and/or prevent development of macular degeneration in a subject in need thereof. In some embodiments, the fluoxetine is substantially enantiomerically pure R-fluoxetine or substantially enantiomerically pure S-fluoxetine. In some embodiments, the subject is a human.

Additionally, it has been determined that administration of racemic fluoxetine preparations to the eye can induce cataract formation. Particularly, cataract formation was observed in up to 50% of subjects to which a racemic fluoxetine preparation was administered. It was surprisingly noted that administration of either enantiomer of fluoxetine did not induce cataract formation.

As such, in some embodiments the presently disclosed subject matter relates to methods for reducing the incidence of cataract formation in subjects receiving fluoxetine, which methods in some embodiments comprise, consist essentially of, or consist of administering the fluoxetine in a substantially pure enantiomeric form rather than as a racemic mixture. Substantially pure enantiomeric form

In some embodiments, the methods of the presently disclosed subject matter are employed as a combination therapy with other active agents that are known to provide benefit for one of the diseases, disorders, and/or conditions for which the presently disclosed subject matter compositions and methods are appropriate.

Thus, in some embodiments the presently disclosed subject matter relates to methods for treating and/or inhibiting progression of a disease, condition, and/or disorder in a subject suffering from and/or at risk for developing a disease, condition, and/or disorder. In some embodiments, the methods comprise administering to the subject a composition comprising a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof, in an amount and via a route effective for treating and/or inhibiting progression of the disease, condition, and/or disorder in the subject. In some embodiments, the disease, condition, and/or disorder comprises wet macular degeneration, dry macular degeneration, a viral infection, acute respiratory distress syndrome, cytokine storm syndrome, fibrosis (e.g., subretinal fibrosis), and/or any combination thereof. In some embodiments, the composition further comprises a pharmaceutically acceptable carrier, diluent, or excipient, optionally a pharmaceutically acceptable carrier, diluent, or excipient that is pharmaceutically acceptable for use in a human. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule. In some embodiments, the methods further comprise administering to the subject an additional treatment. In some embodiments, the additional treatment is a treatment designed to treat or reduce the progression of wet macular degeneration, dry macular degeneration, a viral infection, optionally wherein the viral infection is with a corona virus, further optionally SARS-CoV and/or SARS-CoV-2, acute respiratory distress syndrome, cytokine storm syndrome, fibrosis (e.g., subretinal fibrosis), and/or any combination thereof.

In some embodiments, the presently disclosed subject matter also relates to methods for inhibiting development of macular degeneration in a subject at risk therefor. In some embodiments, the methods comprise administering to the subject a composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for treating and/or inhibiting development of macular degeneration in the subject. In some embodiments, the composition further comprises a pharmaceutically acceptable carrier, diluent, and/or excipient, optionally wherein the composition is pharmaceutically acceptable for use in a human. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule. In some embodiments. The presently disclosed methods further comprising administering to the subject an additional treatment. In some embodiments, the additional treatment is an additional treatment for wet macular degeneration, dry macular degeneration, and/or fibrosis (e.g., subretinal fibrosis).

In some embodiments, the presently disclosed subject matter also relates to methods for inhibiting a viral infection, optionally a viral infection with a coronavirus, further optionally a viral infection with SARS-CoV or SARS-CoV-2. In some embodiments, the methods comprise administering to a subject in need thereof a composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for inhibiting a viral infection in the subject. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule. In some embodiments, the presently disclosed methods further comprise administering to the subject an additional treatment. In some embodiments, the additional treatment is selected from the group consisting of treatment with one or more additional antivirals.

In some embodiments, the presently disclosed subject matter also relates to compositions for use in treating and/or inhibiting development and/or progression of macular degeneration in a subject suffering from and/or at risk for developing macular degeneration. In some embodiments, the compositions comprise, consist essentially of, or consist of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount effective for treating and/or inhibiting development and/or progression of the macular degeneration in the subject.

In some embodiments, the presently disclosed subject matter also relates to compositions for use in treating and/or inhibiting development and/or progression of a viral infection and/or a disease, condition, disorder, and/or symptom associated therewith in a subject suffering from and/or at risk for developing viral infection and/or the disease, condition, disorder, or symptom associated therewith. In some embodiments, the compositions comprise, consist essentially of, or consist of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount effective for treating and/or inhibiting development and/or progression of the viral infection and/or the disease, condition, disorder, and/or symptom associated therewith in the subject.

In some embodiments of the presently disclosed compositions, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule. In some embodiments, the presently disclosed compositions further comprise an additional active agent. In some embodiments, the additional active agent is an agent appropriate for treating macular degeneration and/or an antiviral.

In some embodiments, the presently disclosed subject matter also relates to pharmaceutical compositions for treating and/or inhibiting development and/or progression of macular degeneration in a subject in need thereof, and/or for inhibiting development and/or progression of viral infection and/or a disease, condition, disorder, and/or symptom associated therewith in a subject in need thereof, and/or for treating and/or inhibiting the development and/or progression of acute respiratory distress syndrome, cytokine storm syndrome, and/or fibrosis (e.g., subretinal fibrosis) in a subject in need thereof. In some embodiments, the pharmaceutical compositions comprise, consist essentially of, or consist of an effective amount of one or more a nucleoside reverse transcriptase inhibitors (NRTIs), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof.

In some embodiments, the presently disclosed subject matter also relates to compositions for preparation of a medicament for treating and/or inhibiting development and/or progression of macular degeneration in a subject in need thereof, and/or for inhibiting development and/or progression of viral infection and/or a disease, condition, disorder, and/or symptom associated therewith in a subject in need thereof, and/or for treating and/or inhibiting the development and/or progression of acute respiratory distress syndrome, cytokine storm syndrome, and/or fibrosis (e.g., subretinal fibrosis) in a subject in need thereof, the pharmaceutical composition comprising, consisting essentially of, or consisting of an effective amount of one or more a nucleoside reverse transcriptase inhibitors (NRTIs), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof.

In some embodiments, the presently disclosed subject matter also relates to methods for treating and/or inhibiting the development and/or progression of acute respiratory distress syndrome, cytokine storm syndrome, and/or fibrosis (e.g., subretinal fibrosis), the method comprising administering to a subject in need thereof a composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for treating and/or inhibiting the development and/or progression of acute respiratory distress syndrome, cytokine storm syndrome, and/or fibrosis (e.g., subretinal fibrosis) in the subject. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), pharmaceutically acceptable salts thereof, and combinations thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule. In some embodiments, the presently disclosed methods further comprise administering to the subject an additional treatment. In some embodiments, the additional treatment is selected from the group consisting of treatment with one or more additional active agents, wherein the one or more active agents are appropriate for treating and/or inhibiting the development and/or progression of acute respiratory distress syndrome, cytokine storm syndrome, and/or fibrosis (e.g., subretinal fibrosis).

In some embodiments, the presently disclosed subject matter also relates to compositions for use in treating and/or inhibiting development and/or progression of acute respiratory distress syndrome, cytokine storm syndrome, and/or fibrosis (e.g., subretinal fibrosis) in a subject suffering from and/or at risk for developing acute respiratory distress syndrome, cytokine storm syndrome, and/or fibrosis (e.g., subretinal fibrosis), the composition comprising, consisting essentially of, or consisting of a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount effective for treating and/or inhibiting development and/or progression of the viral infection and/or the disease, condition, disorder, and/or symptom associated therewith in the subject. In some embodiments, the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or an alkylated derivative as set forth in U.S. Pat. No. 10,294,220 (e.g., a Kamuvudine), or any combination thereof. In some embodiments, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule. In some embodiments, the presently disclosed compositions further comprise an additional active agent. In some embodiments, the additional active agent is an agent appropriate for treating acute respiratory distress syndrome, cytokine storm syndrome, and/or fibrosis (e.g., subretinal fibrosis).

Thus, in some embodiments the presently disclosed subject matter relates to compositions and methods for treating and/or inhibiting development and/or progression of a disease, condition, and/or disorder in a subject suffering from and/or at risk for developing a disease, condition, and/or disorder, the method comprising administering to the subject a composition comprising a nucleoside reverse transcriptase inhibitor (NRTI), an alkylated derivative thereof, optionally an alkylated derivative as set forth in U.S. Pat. No. 10,294,220 (e.g., a Kamuvudine), a prodrug thereof, an NRTI metabolite, a pharmaceutically acceptable salt of an NRTI or metabolite thereof, a prodrug of an NRTI, or any combination thereof, in an amount and via a route effective for treating and/or inhibiting progression of the disease, condition, and/or disorder in the subject. In some embodiments, the disease, condition, and/or disorder is macular degermation, including wet macular degeneration and dry macular degeneration. In some embodiments, the disease, condition, and/or disorder is a viral infection, including but not limited to an infection with a coronavirus such as but not limited to SARS-CoV and SARS-CoV-2.

In some embodiments, a reverse transcriptase inhibitor is a nucleoside reverse transcriptase inhibitor (NRTI). Exemplary NRTIs include abacavir ((1S,4R)-4-[2-amino-6-(cyclopropylamino)-9H-purin-9-yl]cyclopent-2-en-1-yl}methanol; ABC; U.S. Pat. No. 8,183,370), adefovir ({[2-(6-amino-9H-purin-9-yl)ethoxy]methyl}phosphonic acid; bis-POM PMEA; U.S. Pat. No. 5,663,159), amdoxovir ([(2R,4R)-4-(2,6-diaminopurin-9-yl)-1,3-dioxolan-2-yl]methanol; Murphy et al. (2010) Antivir Ther 15(2):185-192), apricitabine (4-amino-1-[(2R,4R)-2-(hydroxymethyl)-1,3-oxathiolan-4-yl]pyrimidin-2(1H)-one; AVX754; PCT International Patent Application Publication No. WO 2014/183147), censavudine (1-[(2R,5R)-5-ethynyl-5-(hydroxymethyl)-2H-furan-2-yl]-5-methylpyrimidine-2,4-dione; U.S. Pat. Nos. 7,589,078; 8,193,165; 9,126,971), didanosine (9-((2R,5S)-5-(hydroxymethyl)tetrahydrofuran-2-yl)-3H-purin-6(9H)-one; DDI; U.S. Pat. Nos. 7,589,078; 8,193,165; 9,126,971), elvucitabine (4-amino-5-fluoro-1-[(2S,5R)-5-(hydroxymethyl)-2,5-dihydrofuran-2-yl]pyrimidin-2-one; U.S. Patent Application Publication No. 2011/0150997), emtricitabine (2′,3′-dideoxy-5-fluoro-3′-thiacytidine 4-amino-5-fluoro-1-[(2R,5S)-2-(hydroxymethyl)-1,3-oxathiolan-5-yl]-1,2-dihydropyrimidin-2-one; FTC; PCT International Patent Application Publication No. WO 2014/176532), entecavir (2-Amino-9-[(1S,3R,4S)-4-hydroxy-3-(hydroxymethyl)-2-methyl-idenecyclopentyl]-1H-purin-6-one; ETV; U.S. Pat. No. 6,627,224), lamivudine (2′,3′-dideoxy-3′-thiacytidine-4-Amino-1-[(2R,5S)-2-(hydroxymethyl)-1,3-oxathiolan-5-yl]-1,2-dihydropyrimidin-2-one; 3TC; U.S. Pat. No. 8,481,554), racivir (4-amino-5-fluoro-1-[(2S,5R)-2-(hydroxymethyl)-1,3-oxathiolan-5-yl]-1,2-dihydropyrimidin-2-one; Otto (2004) Curr Opin Pharmacol 4(5):431-436), stampidine (methyl N-((4-bromophenoxy){[(2S,5R)-5-(5-methyl-2,4-dioxo-3,4-dihydropyrimidin-1(2H)-yl)-2,5-dihydrofuran-2-yl]methoxy phosphoryl)-D-alaninate; U.S. Pat. No. 6,350,736), stavudine (1-[(2R,5S)-5-(hydroxymethyl)-2,5-dihydrofuran-2-yl]-5-methyl-1,2,3,4-tetrahydropyrimidine-2,4-dion; d4T; U.S. Pat. No. 8,026,356), tenofovir disoproxil (Bis{[(isopropoxycarbonyl)oxy]methyl}({[(2R)-1-(6-amino-9H-purin-9-yl)-2-propanyl]oxy}methyl)phosphonate; TDF; PCT International Patent Application Publication No. WO 2008/007382), tenofovir alafenamide (Isopropyl (2S)-2-[[[(1R)-2-(6-aminopurin-9-yl)-1-methyl-ethoxy]methyl-phenoxy-phosphoryl]amino]propanoate; GS-7340; U.S. Pat. No. 9,296,769), zalcitabine (4-amino-1-((2R,5S)-5-(hydroxymethyl)tetrahydrofuran-2-yl)pyrimidin-2(1H)-one; ddC; Shelton et al. (1993) Ann Pharmacother 27(4):480-489), zidovudine (ZDV)/azidothymidine (3′-deoxy-3′-azido-thymidine 1-[(2R,4S,5S)-4-Azido-5-(hydroxymethyl)oxolan-2-yl]-5-methylpyrimidine-2,4-dione; AZT; U.S. Pat. Nos. 5,905,082; 6,294,540; 6,417,191), derivatives thereof, optionally alkylated derivatives thereof, further optionally tri-methoxy-3TC (also known as Kamuvudine-9 and K-9) and/or di-ethoxy-AZT (also known as Kamuvudine-8 and K-8) and/or an alkylated derivative as set forth in U.S. Pat. No. 10,294,220 (e.g., a Kamuvudine), pharmaceutically acceptable salts thereof, and combinations thereof. See also U.S. Patent Application Publication Nos. 2019/0022115, 2019/0055273, 2019/0177326, 2019/0185508. Each of these U.S. Patents and Patent Applications publications is incorporated by reference in its entirety.

In some embodiments of the presently disclosed methods, the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof. In some embodiments, the composition is formulated for administration in a depot and/or for sustained release. In some embodiments, the composition is formulated in a targeted drug delivery system, optionally as part of a nanoparticle and/or a microparticle, further optionally a liposome, wherein the nanoparticle and/or a microparticle comprises a targeting molecule, optionally a tissue-specific antibody.

In some embodiments, suitable formulations can also include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The methods of the presently disclosed subject matter can be used with additional adjuvants or biological response modifiers including, but not limited to, cytokines and other immunomodulating compounds.

In some embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the compositions described herein. Drugs useful in the presently disclosed subject matter may, for example, possess a pharmaceutical property selected from the group consisting of antimitotic, antikinase, alkylating, antimetabolite, antibiotic, alkaloid, anti-angiogenic, pro-apoptotic agents, and combinations thereof.

The compositions of the presently disclosed subject matter can be administered by any route of administration reasonably expected to deliver the compositions to a desired target site. Suitable methods for administration of the compositions of the presently disclosed subject matter thus include, but are not limited to intravenous administration and delivery directly to the target tissue or organ. In some embodiments, the method of administration encompasses features for regionalized delivery or accumulation of the composition at the site in need of treatment. In some embodiments, the composition is/are delivered directly into the nervous system. In some embodiments, selective delivery of the composition is accomplished by intravenous injection of composition, where they accumulate in the nervous system (e.g., the brain). Other modes of administration that can be employed include topical, oral, buccal, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means. Compounds or agents of the presently disclosed subject matter can be administered to a subject by one or more of these routes when appropriate.

Where the administration of the composition is by injection or direct application, the injection or direct application may be in a single dose or in multiple doses. Where the administration of the composition is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient(s), the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, ed., 1985, Remington's Pharmaceutical Sciences, Mack Publishing Co., Easton, Pa., United States of America, which is incorporated herein by reference.

An effective dose of a composition of the presently disclosed subject matter is administered to a subject in need thereof. A “treatment effective amount”, “therapeutic amount”, or “therapeutically effect amount” is an amount of a therapeutic composition sufficient to produce a measurable response (e.g., a biologically or clinically relevant response in a subject being treated). In some embodiments, an activity that inhibits a viral infection is measured. Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using generally applicable assay methods, one skilled in the art can readily assess the potency and efficacy of a candidate compound of the presently disclosed subject matter and adjust the therapeutic regimen accordingly. After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

As such, in some embodiments the presently disclosed composition thereof is/are present in a pharmaceutically acceptable carrier, which in some embodiments can be a pharmaceutically acceptable for use in humans.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In some embodiments, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the active agent(s) encapsulated in nanoparticles and/or microparticles (including but not limited to liposomes). The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

Compositions and methods for encapsulating active agents in nanoparticles and/or microparticles (including but not limited to liposomes) are disclosed, for example, in U.S. Pat. No. 9,867,888 and U.S. Patent Application Publication Nos. 2018/0140717, 2018/0147298, 2018/0148719, 2018/0177727, 2018/0221402, and 2019/0345492, each of which is incorporated herein by reference in its entirety.

In some embodiments, the presently disclosed subject matter provides use of pharmaceutical compositions comprising, consisting essentially of, or consisting of an effective amount of a composition comprising one or more a reverse transcriptase inhibitors to treat and/or inhibit development and/or progression of macular degeneration, either wet or dry macular degeneration, and/or conditions, and/or disorders associated with a viral infection, optionally a coronavirus infection, further optionally an infection with SARS-CoV and/or SARS-CoV-2 in a subject in need thereof.

In some embodiments, the presently disclosed subject matter provides use of an effective amount of a composition comprising one or more a reverse transcriptase inhibitors for the preparation of a medicament to treat and/or inhibit development and/or progression of macular degeneration, either wet or dry macular degeneration, and/or conditions, and/or disorders associated with a viral infection, optionally a coronavirus infection, further optionally an infection with SARS-CoV and/or SARS-CoV-2 in a subject in need thereof.

The presently disclosed subject matter also relates in some embodiments to compositions for use in the presently disclosed methods. In some embodiments, the compositions comprise an amount effective for treat and/or inhibit development and/or progression of macular degeneration, either wet or dry macular degeneration, and/or conditions, and/or disorders associated with a viral infection, optionally a coronavirus infection, further optionally an infection with SARS-CoV and/or SARS-CoV-2 in a subject in need thereof.

In some embodiments, the presently disclosed compositions and uses comprise, consist essentially of, and/or consist of a nucleoside reverse transcriptase inhibitor (NRTI), an NRTI metabolite, a pharmaceutically acceptable salt of an NRTI or metabolite thereof, a prodrug of an NRTI, or any combination thereof. Any other NRTIs, metabolites thereof, pharmaceutically acceptable salts thereof, prodrugs thereof, whether known or not yet developed can be employed in the compositions of the presently disclosed subject matter.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for the EXAMPLES

Animals. Both male and female wild-type (WT) C57BL/6J mice (The Jackson Laboratory, Bar Harbor, Me., United States of America) between 6-10 weeks of age were used in this study. For all procedures, anesthesia was achieved by intraperitoneal injection of 100 mg/kg ketamine hydrochloride (Fort Dodge Animal Health, Overland Park, Kans., United States of America) and 10 mg/kg xylazine (Phoenix Scientific, Inc., St. Joseph, Mo., United States of America), and pupils were dilated with topical 1% tropicamide and 2.5% phenylephrine hydrochloride (Alcon Laboratories, Inc., Ft. Worth, Tex., United States of America). All animal experiments were approved by the Institutional Animal Care and Use Committees of the University of Virginia (Charlottesville, Va., United States of America) and was performed in accordance with the Statement for the Use of Animals in Ophthalmic and Visual Research of the Association for Research in Vision and Ophthalmology (Washington, D.C., United States of America).

Subretinal and intravitreous injections. Subretinal injections (SRI) (1 μl) and intravitreous injections (0.5 μl) in mice were performed using a 35-gauge needle (Ito Co.). In vitro transcribed Alu RNA (300 ng/μl) was injected into the subretinal space via SRI. 1 mM of 3′-Azido-3′-deoxythymidine β-D-glucuronide (GAZT; Cayman Chemical, Ann Arbor, Mich., United States of America), Lamivudine (R)-sulfoxide (LSO; Toronto Research Chemicals, Toronto, Canada) 2 mM of 3′-Amino-3′-deoxythymidine (AMT; Toronto Research Chemicals), or PBS was injected into the vitreous humor. Kamuvudines including but not limited to K-8 (2-Et-AZT) and K-9 (3-Me-3TC, a.k.a. 3-Me-lamivudine) were previously described (see U.S. Pat. No. 10,294,220, which is incorporated herein by reference in its entirety).

Choroidal neovascularization. Experimental choroidal neovascularization (CNV) was induced via laser photocoagulation (OcuLight GL, IRIDEX Corp., Mountain View, Calif., United States of America; 180 mW, 100 ms, 75 μm) in both eyes. The volume of the CNV lesions was measured after 7 days using a scanning laser confocal microscope (A1R Nikon confocal microscope system, Nikon USA, Melville, N.Y., United States of America) after 0.7% FITC-isolectin B4 staining (Vector Laboratories, Burlingame, Calif., United States of America, USA). The volume of each lesion was calculated using FIJI software (Schindelin et al., 2012).

Assessment of RPE Degeneration. Seven days after SRI, RPE health was assessed by fundus photography and immunofluorescence staining of zonula occludens-1 (ZO-1) on retinal pigmented epithelium (RPE) flat mounts (whole mount of posterior eye cup containing RPE and choroidal layers). Mouse RPE and choroidal flat mounts were fixed with 2% paraformaldehyde, stained with rabbit polyclonal antibodies against mouse ZO-1 (1:100; Invitrogen, Carlsbad, Calif., United States of America) and visualized with Alexa-594 (Invitrogen). All images were obtained by confocal microscopy (model A1R Nikon confocal microscope system, Nikon USA). Imaging was performed by an operator blinded to the group assignments.

Subretinal fibrosis. Subretinal fibrosis was induced via laser using the same protocol for experimental CNV. The eyes were collected after 7 days and stained for collagen type I using rabbit anti-collagen type I antibody (1:100; Rockland Immunochemicals, Limerick, Pa., United States of America). The secondary antibody was Alexa 555 (1:200; Thermo Fisher Scientific). After mounting in slides, the RPE flat mounts were imaged using a scanning laser confocal microscope (A1R Nikon confocal microscope system, Nikon USA) and the volume of each lesion was calculated by FIJI software.

Fundus photography. Fundus imaging of dilated mouse eyes was performed using a TRC-50 IX camera (Topcon, Oakland, N.J., United States of America) linked to a digital imaging system (Sony, New York, N.Y., United States of America).

Cell Stimulation. Bone marrow derived macrophages (BMDMs) were treated with 200 μM Zidovudine (AZT), GAZT, AMT, or LSO for 1 hour. Cells were transfected with Alu RNA overnight using Lipofectamine 3000 (Invitrogen) according to manufacturer's instructions.

Western blotting. Serum free media (1 mL) from stimulated BMDMs was transferred into fresh tubes and centrifuged for 10 min at 12,000 rpm at 4° C. 15 μl of 10% sodium deoxycholate was added to the supernatants, vortexed and kept on ice. 72 μl Trichloroacetic acid (Sigma-Aldrich, St. Louis,. Mo., United States of America) was subsequently added and the tubes were kept on ice overnight. After centrifugation for 30 min at 12,000 rpm at 4° C., the pellet was washed twice with 500 μl ice-cold acetone. The pellet was dried and boiled with LDS Sample Buffer and β-mercaptoethanol (Sigma-Aldrich). The precipitated protein was analyzed by immunoblotting.

Enzyme-linked immunosorbent assay (ELISA). 1.2 million cells/mL were seeded per well in 6-well cell culture dishes overnight and stimulated as mentioned above. Secreted mouse IL-1β in the medium were detected by ELISA (R&D Systems, Inc., Minneapolis, Minn., United States of America) according to the manufacturer's instructions.

### Example 1

### NRTI and NRTI Metabolites Inhibited Inflammasome Activation

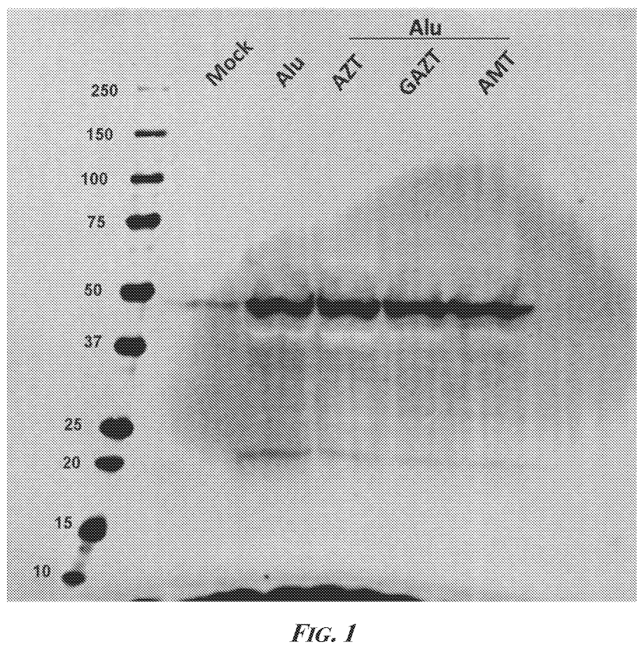

The ability of NRTI and NRTI metabolites to inhibit inflammasome activation was tested by western blot. The results are shown in FIG. 1.

As shown FIG. 1 is a representative western blot showing Alu RNA-induced cleavage of pro-caspase-1 (50 kDa band) into active caspase-1 (20 kDa band), a hallmark of inflammasome activation, was inhibited by azidothymidine (AZT), its metabolite azidothymidine glucuronide (GAZT), and a further metabolite 3′-amino-3′-deoxythymidine (AMT) in mouse BMDMs.

### Example 2

### NRTI and NRTI Metabolites Inhibited IL-1β Release

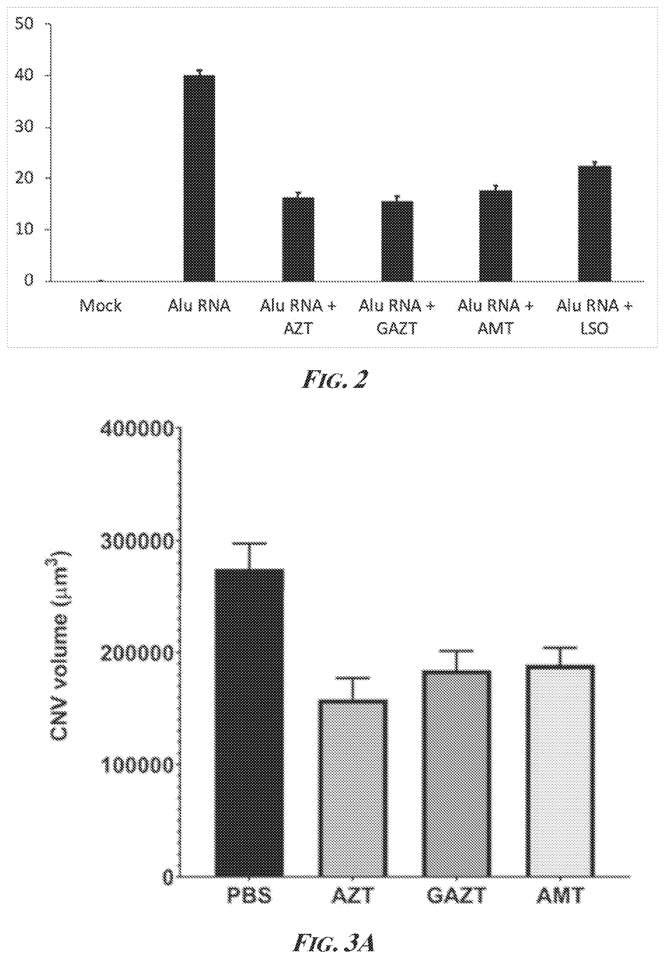

The ability of NRTI and NRTI metabolites to inhibit IL-1β release was tested by ELISA. The results are shown in FIG. 2.

As shown in FIG. 2, Alu RNA-induced IL-1β release, as monitored by ELISA, from mouse BMDMs was inhibited by AZT, GAZT, AMT, and LSO.

### Example 3

### NRTI, NRTI Metabolites, and NRTI Analogs Inhibited a Mouse Model of Neovascular (“Wet”) Macular Degeneration

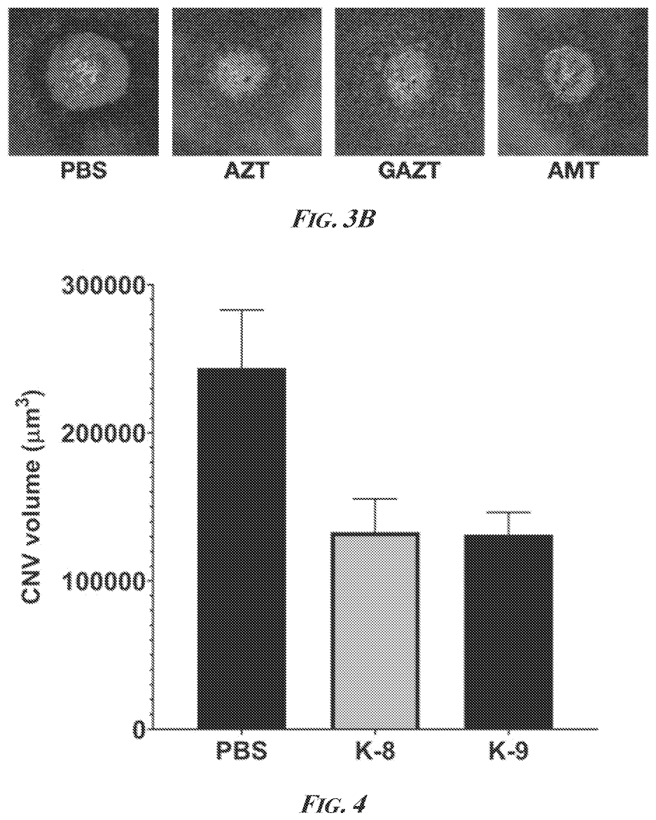

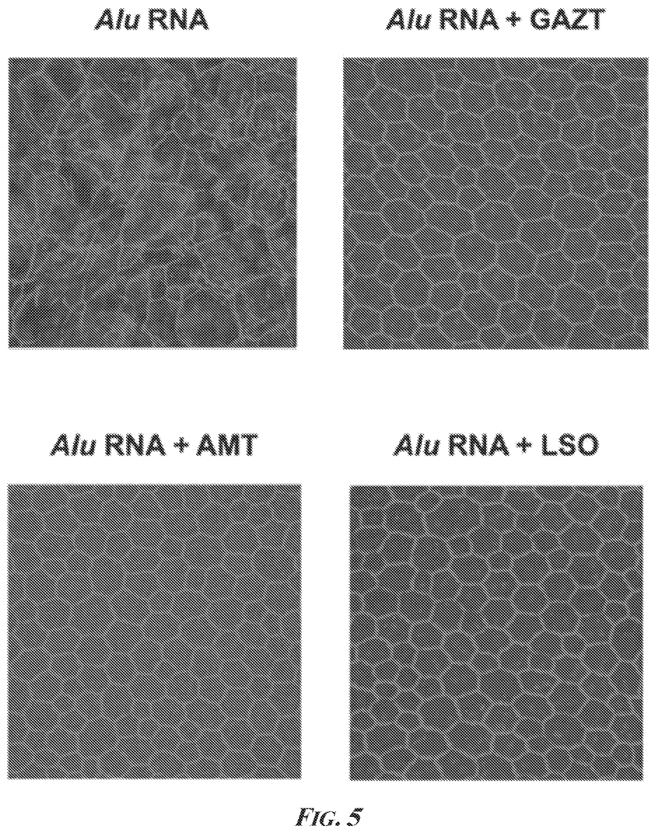

The ability of NRTI, NRTI metabolites, and NRTI analogs to inhibit neovascular (wet) macular degeneration in a mouse model was tested. The results are shown in FIGS. 3A and 3B and in FIG. 4.

FIGS. 3A and 3B are a bar graph and a fluorescence micrograph, respectively, of the results of laser-induced choroidal neovascularization (CNV) in mice, which was inhibited by AZT, GAZT, and AMT. FIG. 3A provides a summary of CNV volumes. FIG. 3B is a series of representative fluorescence images of CNV lesions.

FIG. 4 is a bar graph showing that laser-induced choroidal neovascularization (CNV) in mice was inhibited by the NRTI analogs K-8 and K-9.

### Example 4

### NRTI and NRTI Metabolites Inhibited a Mouse Model of Atrophic (“Dry”) Macular Degeneration

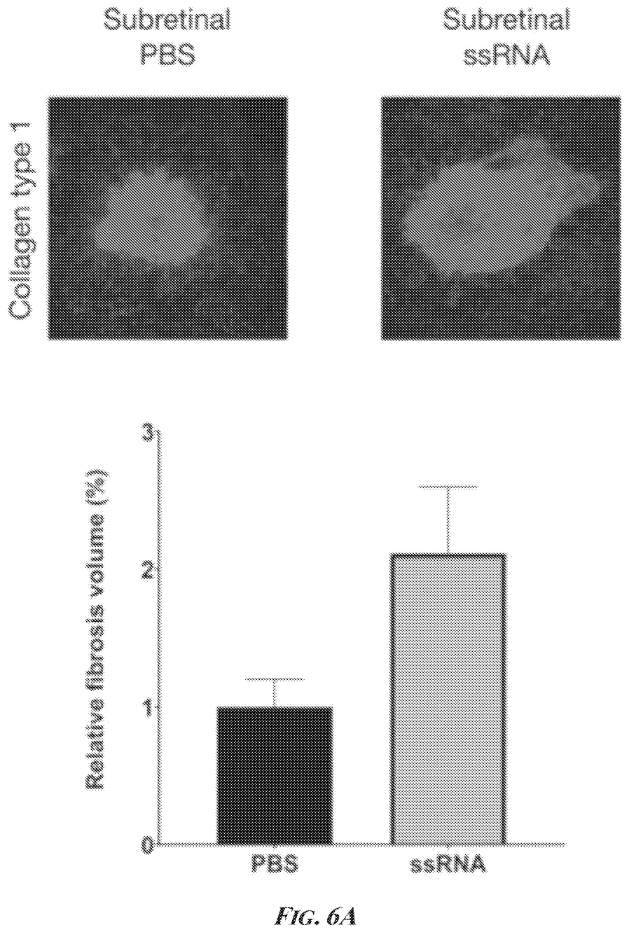

The ability of NRTI and NRTI metabolites to inhibit atrophic (dry) macular degeneration in a mouse model was also tested. The results are shown in FIG. 5.

FIG. 5 is a series of fluorescence micrographs of representative flat mounts of the retinal pigmented epithelium (RPE) of mice showing that Alu RNA-induced RPE degeneration was inhibited by GAZT, AMT, and LSO.

### Example 5

### NRTI and the NRTI Analog K-8 Inhibited a Mouse Model of Fibrosis

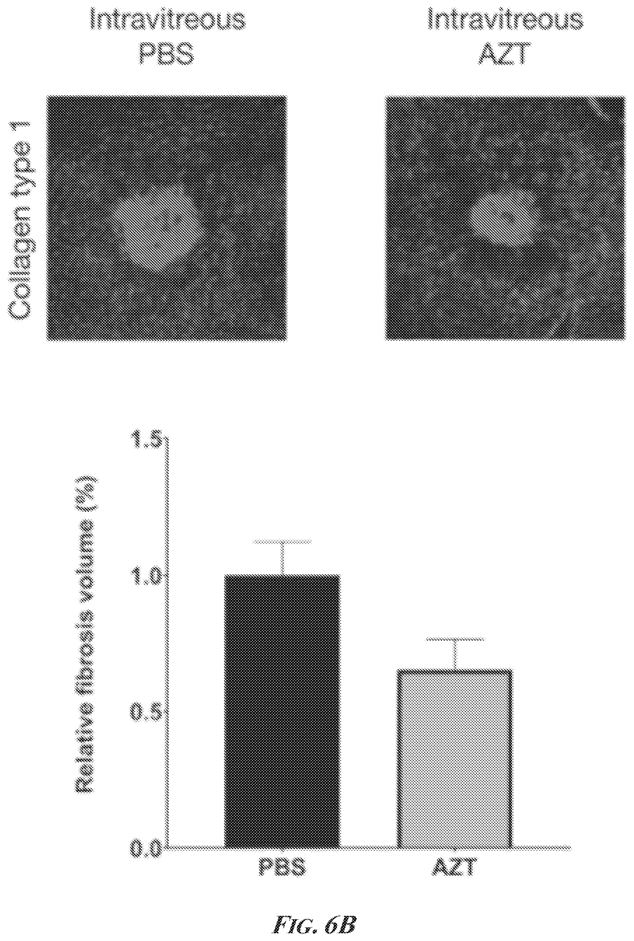

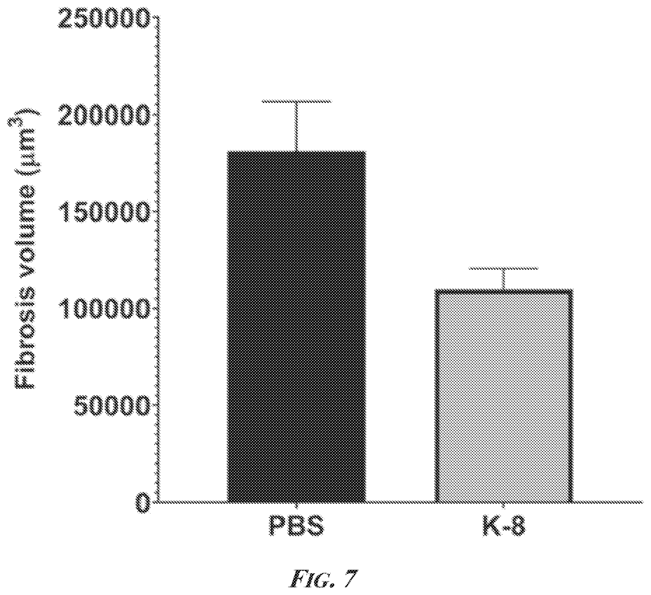

The ability of NRTI and the NRTI analog K-8 to inhibit fibrosis in a mouse model was tested. The results are shown in FIGS. 6 and 7.

FIGS. 6A and 6B present the results of experiments showing that NRTI blocked Alu RNA-induced fibrosis. FIG. 6A is a pair of fluorescence micrographs (top panels) and a bar graph (bottom panel) showing that wild type mice injected with subretinal Alu RNA after laser injury displayed a significantly larger volume of subretinal fibrosis than mice injected with PBS. FIG. 6B is a pair of fluorescence micrographs (top panels) and a bar graph (bottom panel) showing that intravitreous injection of the NRTI AZT reduced subretinal fibrosis volume in wild type mice. Images are representative of flat mounts stained with an anti-collagen type 1 antibody to visualize fibrosis.

FIG. 7 is a bar graph showing that the NRTI analog K-8 also blocked Alu RNA-induced fibrosis. Intravitreous injection of the NRTI analog K-8 reduced the volume of subretinal fibrosis induced by subretinal Alu RNA after laser injury in wild-type mice. Images are representative of flat mounts stained with an anti-collagen type 1 antibody to visualize fibrosis.

### Discussion of the EXAMPLES

Here, we report that three human drug metabolites of FDA-approved NRTIs inhibit activation of the NLRP3 inflammasome: they inhibit cleavage of pro-caspase-1 to caspase-1 and inhibit secretion of the cytokine IL-1β. We also demonstrate that NRTI, NRTI metabolites, and NRTI analogs inhibit RPE degeneration in an in vivo model of dry macular degeneration, inhibit angiogenesis in an in vivo model of wet macular degeneration, and inhibit fibrosis in an in vivo model of subretinal fibrosis.

The ongoing coronavirus disease 2019 (COVID-19) pandemic is caused by the severe acute respiratory syndrome coronavirus 2 (SARS-CoV-2), a single-stranded RNA (ssRNA) virus (Zhou et al., 2020). Cytokine storm syndrome (CSS) and acute respiratory distress syndrome (ARDS) are principal causes of mortality in patients with COVID-19, and are characterized by NLRP3 inflammasome activation, epithelial damage, endothelial dysfunction, and fibrosis. These cellular and tissues pathologies are triggered by ssRNA Alu RNA as well. Collectively, our studies indicate that NRTIs, NRTI metabolites, and NRTI analogs are potential candidates for the treatment of dry macular degeneration, wet macular degeneration, and inflammatory pathologies associated with viral diseases such as COVID-19.

Accordingly, disclosed herein is the discovery that human drug metabolites of FDA-approved NRTIs possess anti-inflammatory, anti-cytotoxic, and anti-angiogenic activity in animal models of atrophic and neovascular macular degeneration. Per FDA regulations (https://www.fda.gov/media/72279/download), these metabolites would have undergone sufficient additional testing to convince the regulators that they are safe for human use. Hence, these metabolites could be fast-tracked for clinical trial testing in both “dry” and “wet” macular degeneration.

NRTIs and NRTI metabolites thus have profound effects on suppressing inflammation, epithelial cell damage, and endothelial dysfunction induced by Alu RNA, which, like severe acute respiratory syndrome coronavirus 2 (SARS-CoV-2), is also an single-stranded RNA (ssRNA) that is capable of evoking host antiviral responses that mimic the cellular response to ssRNA viruses. Therefore, these drugs could also be candidates for treating cytokine storm syndrome (CSS) and acute respiratory distress syndrome (ARDS) in patients with coronavirus disease 2019 (COVID-19), which is caused by SARS-CoV-2.

The absence of CSS is associated with rapid and unremarkable recovery among COVID-19 patients (Thevarajan et al., 2020). An exuberant innate immune response to ssRNAs is one of the earliest signaling pathways responsible for the cytokine storm; blunting this response has been identified as a treatment strategy for CSS (Teijaro et al., 2014). Data on SARS-CoV-2 and influenza-associated ARDS suggest IL-1β to be a key cytokine driving the pro-inflammatory activity in patients with lung injury (Tisoncik et al., 2012). The histopathology of ARDS includes epithelial damage, endothelial dysfunction, edema, and inflammation (Ware & Matthay, 2000; Wheeler & Bernard, 2007). Collectively, given the aforementioned data that NRTIs and their metabolites impair multiple ill effects of ssRNA exposure on inflammation, epithelial damage, endothelial dysfunction, and fibrosis, these drugs could be beneficial for CSS and ARDS in COVID-19.

### REFERENCES

All references listed below, as well as all other references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.

Altschul et al. (1990a) Basic local alignment search tool. J Mol Biol 215:403-410.
Altschul et al. (1990b) Protein database searches for multiple alignments. Proc Natl Acad Sci USA 87:14:5509-13.
Altschul et al. (1997) Gapped BLAST and PSI-BLAST: a new generation of protein database search programs. Nucleic Acids Res 25:3389-3402.
Bird et al. (1988) Single-chain antigen-binding proteins. Science 242:423-426.
Devereux et al. (1984) A comprehensive set of sequence analysis programs for the VAX. Nucl. Acids Res., 12:387-395.
Fowler et al. (2014) Nucleoside reverse transcriptase inhibitors possess intrinsic anti-inflammatory activity. Science 346:1000-3.

### Genaro (ed.) (1985) Remington's Pharmaceutical Sciences, Mack Publishing Co., Easton, Pa., United States of America.

### Gross & Mienhofer (1981) The Peptides, Vol. 3, Academic Press, New York, N.Y., United States of America, pp. 3-88.

### Harlow & Lane (1988) Antibodies, A Laboratory Manual, Cold Spring Harbor Laboratory Publications, Cold Spring Harbor, New York, United States of America.

Huston et al. (1988) Protein engineering of antibody binding sites: recovery of specific activity in an anti-digoxin single-chain Fv analogue produced in Escherichia coli. Proc Natl Acad Sci U S A 85:5879-5883.
Kaneko et al. (2011) DICER1 deficit induces Alu RNA toxicity in age-related macular degeneration. Nature 471:325-30.
Kerur et al. (2018) cGAS drives noncanonical-inflammasome activation in age-related macular degeneration. Nat Med 24:50-61.
Mitchell et al. (2018) Age-related macular degeneration. Lancet 392:1147-59.

### Mizutani et al. (2015) Invest Ophthalmol Vis Sci. 56:7122-9.

Otto (2004) New nucleoside reverse transcriptase inhibitors for the treatment of HIV infections. Curr Opin Pharmacol 4(5):431-436
PCT International Patent Application Publication Nos. WO 2008/007382, WO 2014/176532, WO 2014/183147.
Schindelin et al. (2012) Fiji: an open-source platform for biological-image analysis. Nature Methods 9(7): 676-682.
Shelton et al. (1993) Zalcitabine. Ann Pharmacother 27(4):480-489.
Tarallo et al. (2012) DICER1 loss and Alu RNA induce age-related macular degeneration via the NLRP3 inflammasome and MyD88. Cell 149:847-859.
Teijaro et al. (2014) Mapping the innate signaling cascade essential for cytokine storm during influenza virus infection. Proc Natl Acad Sci U S A. 111:3799-804.
Thevarajan et al. (2020) Breadth of concomitant immune responses prior to patient recovery: a case report of non-severe COVID-19. Nat Med. Online Publication Mar. 16, 2020. https://doi.org/10.1038/s41591-020-0819-2.
Tisoncik et al. (2012) Into the Eye of the Cytokine Storm. Microbiol Mol Biol Rev. 76:16-32.
U.S. Patent Application Publication Nos. 2003/0017534; 2011/0150997; 2016/138425; 2018/0140717; 2018/0147298; 2018/0148719; 2018/0177727; 2018/0221402; 2018/0298087; 2018/0312588; 2018/0346564; 2019/0022115; 2019/0055273; 2019/0151448; 2019/0177326; 2019/0185508; 2019/0262341; 2019/0345492.
U.S. Pat. Nos. 4,816,567; 5,482,856; 5,663,159; 5,905,082; 5,922,695; 6,294,540; 6,350,736; 6,358,527; 6,417,191; 6,479,284; 6,627,224; 6,677,436; 7,060,808; 7,589,078; 7,906,625; 8,026,356; 8,183,370; 8,193,165; ,398,980; 8,436,150; 8,481,554; 8,796,439; 9,126,971; 9,296,769; 9,867,888; 10,253,111; 10,294,220.
Ware & Matthay (2000) The acute respiratory distress syndrome. N Engl J Med 342:1334-49.
Wheeler & Bernard (2007) Acute lung injury and the acute respiratory distress syndrome: a clinical review. Lancet 369:1553-64.
Wong et al. (2014) Global prevalence of age-related macular degeneration and disease burden projection for 2020 and 2040: a systematic review and meta-analysis. Lancet Global Health 2:e106-16.
Zhou et al. (2020) A pneumonia outbreak associated with a new coronavirus of probable bat origin. Nature. 579:270-3.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating and/or inhibiting progression of a disease, condition, and/or disorder in a subject suffering from and/or at risk for developing a disease, condition, and/or disorder, the method comprising administering to the subject a composition comprising a nucleoside reverse transcriptase inhibitor (NRTI), an alkylated derivative thereof, optionally wherein the alkylated derivative is a Kamuvudine, a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof, in an amount and via a route effective for treating and/or inhibiting progression of the disease, condition, and/or disorder in the subject.

2. The method of claim 1, wherein the disease, condition, and/or disorder comprises wet macular degeneration, dry macular degeneration, a viral infection, acute respiratory distress syndrome, cytokine storm syndrome, fibrosis, and/or any combination thereof.

3. The method of claim 1, wherein the composition further comprises a pharmaceutically acceptable carrier, diluent, or excipient, optionally a pharmaceutically acceptable carrier, diluent, or excipient that is pharmaceutically acceptable for use in a human.

4. The method of claim 3, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or optionally an alkylated derivative thereof, further optionally wherein the alkylated derivative is a Kamuvudine, or any combination thereof.

5. The method of claim 1, wherein the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof.

6. The method of claim 1, wherein the composition is formulated for administration in a depot and/or for sustained release.

7. The method of claim 1, wherein the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

8. The method of claim 1, further comprising administering to the subject an additional treatment.

9. The method of claim 8, wherein the additional treatment is a treatment designed to treat or reduce the progression of wet macular degeneration, dry macular degeneration, a viral infection, optionally wherein the viral infection is with a corona virus, further optionally SARS-CoV and/or SARS-CoV-2, acute respiratory distress syndrome, cytokine storm syndrome, fibrosis, and/or any combination thereof.

10. A method for inhibiting development of macular degeneration in a subject at risk therefor, the method comprising administering to the subject a composition comprising, consisting essentially of, or consisting of a nucleoside reverse transcriptase inhibitor (NRTI), an alkylated derivative thereof, optionally wherein the alkylated derivative is a Kamuvudine, a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for treating and/or inhibiting development of macular degeneration in the subject.

11. The method of claim 10, wherein the composition further comprises a pharmaceutically acceptable carrier, diluent, and/or excipient, optionally wherein the composition is pharmaceutically acceptable for use in a human.

12. The method of claim 11, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or a Kamuvudine, or any combination thereof.

13. The method of claim 10, wherein the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof.

14. The method of claim 10, wherein the composition is formulated for administration in a depot and/or for sustained release.

15. The method of claim 10, wherein the composition is formulated in a drug delivery system, wherein the drug delivery system optionally comprises a bioerodible implant, a biodegradable implant, a nanoparticle, and/or a microparticle, further optionally a liposome, wherein the nanoparticle and/or a microparticle optionally further comprises a targeting molecule.

16. The method of claim 10, further comprising administering to the subject an additional treatment.

17. The method of claim 16, wherein the additional treatment is an additional treatment for wet macular degeneration, dry macular degeneration, and/or fibrosis.

18. A method for inhibiting a viral infection, optionally a viral infection with a coronavirus, further optionally a viral infection with SARS-CoV or SARS-CoV-2, the method comprising administering to a subject in need thereof a composition comprising, consisting essentially of, or consisting of a nucleoside reverse transcriptase inhibitor (NRTI), an alkylated derivative thereof, optionally wherein the alkylated derivative is a Kamuvudine, a prodrug thereof, an NRTI metabolite, optionally wherein the NRTI metabolite is selected from the group consisting of 3′-Azido-3′-deoxythymidine β-D-glucuronide (GAZT), 3′-Amino-3′-deoxythymidine (AMT), and Lamivudine (R)-sulfoxide (LSO), a pharmaceutically acceptable salt thereof, or any combination thereof in an amount and via a route effective for inhibiting a viral infection in the subject.

19. The method of claim 18, wherein the NRTI is selected from the group consisting of abacavir (ABC), adefovir (bis-POM PMEA), amdoxovir, apricitabine (AVX754), censavudine, didanosine (DDI), elvucitabine, emtricitabine (FTC), entecavir (ETV), lamivudine (3TC), racivir, stampidine, stavudine (d4T), tenofovir disoproxil (TDF), tenofovir alafenamide (GS-7340), zalcitabine (ddC), zidovudine (ZDV)/azidothymidine (AZT), derivatives thereof, optionally alkylated derivatives thereof, further optionally tri-methoxy-3TC and/or di-ethoxy-AZT and/or a Kamuvudine, pharmaceutically acceptable salts thereof, and combinations thereof.

20. The method of claim 18, wherein the composition is formulated for administration orally, rectally, topically, by aerosol, by injection, parenterally, intramuscularly, subcutaneously, intravenously, intramedullarily, intrathecally, intraventricularly, intraperitoneally, intranasally, intraocularly, intracranially, or any combination thereof.

21-48. (canceled)
